(19) United States
(12) Patent Application Publication
(10) Pub. No.: US 2017/0354972 A1
(43) Pub. Date: Dec. 14, 2017

(54) Systems and Methods for Determining Probative Samples and Isolation and Quantitation of Cells

(71) Applicants: Leonard Klevan, Cave Creek, AZ (US); Fatih Inci, Palo Alto, CA (US); Utkan Demirci, Stanford, CA (US)

(72) Inventors: Leonard Klevan, Cave Creek, AZ (US); Fatih Inci, Palo Alto, CA (US); Utkan Demirci, Stanford, CA (US)

(21) Appl. No.: 15/522,232

(22) PCT Filed: Sep. 30, 2015
(86) PCT No.: PCT/US2015/053370

§ 371 Date: Apr. 26, 2017

Related U.S. Application Data
(60) Provisional application No. 62/058,072, filed on Sep. 30, 2014.

(51) Int. Cl. B01L 3/00; G01N 1/40; C12Q 1/24

(52) U.S. Cl. CPC B01L 3/502761; C12Q 1/24; G01N 1/405; B01L 2200/0668; B01L 2300/0636; B01L 2300/0816

(57) ABSTRACT

Embodiments of the present disclosure relate to a platform for at least one of capturing, identifying and studying biological materials, and more particularly, to microfluidic channel platforms (for example) for detecting and/or identifying samples containing sperm cells, and isolating and analyzing captured sperm cells for DNA analysis (for example). In some embodiments, such microfluidic platforms integrate imaging technology. Such embodiments provide the ability to at least one of rapidly isolate and quantitate sperm cells from biological mixtures as occur in sexual assault evidence, for example, thereby enhancing identification of suspects in these cases and contributing to the safety of society.

### CROSS REFERENCES TO RELATED APPLICATIONS

This patent application claims priority to U.S. Provisional Patent Application No. 62/058,072, filed Sep. 30, 2014, and titled “Systems and Methods for Selective Isolation and Quantitation of Cells”, the entire disclosure of which is herein incorporated by reference.

### GOVERNMENT RIGHTS

This invention was made with Government support under award no. 1464673 awarded by the National Science Foundation. The Government has certain rights in this invention.

### FIELD OF THE INVENTION

The present disclosure relates to platforms for at least one of capturing, identifying and studying biological materials, and more particularly, to microfluidic channel platforms (for example) for detecting and/or identifying samples containing sperm cells, and isolating and analyzing captured sperm cells for DNA analysis (for example). In some embodiments, such microfluidic platforms integrate imaging technology. Such embodiments provide the ability to at least one of rapidly isolate and quantitate sperm cells from biological mixtures as occur in sexual assault evidence, for example, thereby enhancing identification of suspects in these cases and contributing to the safety of society.

### BACKGROUND

Although forensic DNA testing has contributed immensely to the successful processing and analysis of evidence materials collected at crime scenes, especially crimes of a sexual nature, the time consuming steps involved in such processes and analysis have led to an immense backlog that has overwhelmed the available capacity of forensic laboratories. For example, it currently takes hours to separate sperm cells from samples, and in some cases, the effort may be a waste of precious resources if the sample does not contain usable cells. Such situations arise due to the lack of reliable methods for identifying useful samples prior to the extraction of the sperm cells.

### SUMMARY OF SOME OF THE EMBODIMENTS

Some embodiments of the present disclosure address problems of the prior art. For example, in some embodiments, a system for capturing and/or detecting target cells in a biological sample are provided and include (for example): one or more microfluidic channels for receiving a biological sample, a recognition reagent linked to a surface of the one or more channels, which may also be referred to as a capture molecule or material that may be linked to the surface of a channel (or other surface, e.g., bead) directly or via another molecule and/or substance (e.g., the term “reagent” or phrase “recognition reagent” can correspond to or be referred to as a capture molecule or material, or similar functionality). The reagent is configured to capture one or more target cells contained in the biological sample by binding with the one or more target cells, and a monitoring means configured to at least one of monitor the surface and detect one or more captured target cells bound with the reagent linked to the surface. The monitoring means can be configured to at least one of receive and collect data on the captured target cells. In some embodiments, the monitoring means may comprise an imaging means configured to acquire images of captured target cells. Additionally (or in place of), such monitoring means may include at least one of mechanical means, electrical means, optical means, photonic means, and plasmonic means. Further, the monitoring means may correspond to or include a smartphone for at least one of image capture and information/image analysis. For example, in some embodiments, the smartphone is equipped with an application configured for receiving and/or collecting data of at least one of the surface (e.g., image information) and data on any captured target cells. In some embodiments, the target cells in the biological sample can be sperm cells, blood cells, bacteria, yeasts, fungi, and/or viruses.

In some embodiments, the recognition reagent comprises an oligosaccharide sequence. The oligosaccharide sequence may comprise a sialyl-Lewis^{x} oligosaccharide sequence. In some embodiments, the microfluidic channels can have dimensions ranging from about 25 micron to about 80 micron.

Some embodiments of the current disclosure are directed to (or further include) methods for capturing and/or detecting target cells in a biological sample. Such methods comprise: providing a surface having linked thereto one or more oligosaccharide molecules, where the oligosaccharide molecules are configured to capture one or more target cells, exposing the surface to a biological sample, capturing one or more target cells contained in the sample, where a target cell is captured by binding with at least one of the oligosaccharide molecules, and at least one of monitoring the surface and detecting the at least one captured target cell. In addition, the steps may further comprise at least one of receiving and collecting data corresponding to at least one of the surface and captured target cells. Further, the steps may include at least one of releasing, lysing, and processing the captured target cells. In some embodiments, the target cells may be sperm cells, blood cells, bacteria, yeasts, fungi, and/or viruses.

In some embodiments, the at least one of monitoring and detecting step may comprise receiving and/or collecting data associated with at least one of the surface and captured target cells bound thereon via the oligosaccharide. For example, the data may be received and/or collected via a monitoring means, and the at least one of receiving and collecting data may comprise imaging the surface and/or bound target cells. In some embodiments, the oligosaccharide molecules may comprise sialyl-Lewis^{x} oligosaccharide molecules. In some embodiments, the step of exposing comprises flowing the biological sample over the surface. The surface may include at least one of the inner surface of one or more microfluidic channels and the surface of one or more beads.

Some embodiments of the current disclosure also include a method for capturing and detecting target cells in a plurality of biological samples, comprising: identifying, via shadow imaging (for example), probative samples for capturing and/or detecting target cells from the plurality of biological samples, providing a surface having linked thereto one or more oligosaccharide molecules, where the oligosaccharide molecules are configured to capture one or more target cells, exposing the surface to the probative samples for capturing and detecting target cells, capturing one or more target cells contained in the probative sample, where a target cell is captured by binding with at least one of the oligosaccharide molecules, and extracting DNA of the target cells contained in the probative sample.

One of skill in the art will appreciate that some embodiments may be configured such that, target cells can be selectively separated from a sample (e.g., for enrichment), and, in some embodiments, non-target cell types can be separated so as to eliminate them from the sample.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings are primarily for illustrative purposes and are not intended to limit the scope of the inventive subject matter described herein. Moreover, the drawings are not necessarily to scale, as, in some instances, various aspects of inventive subject matter may be shown exaggerated or enlarged to facilitate an understanding of different features. Additionally, in the drawings, like reference characters generally refer to like features (e.g., functionally similar and/or structurally similar elements).

FIG. 1 shows an example flow diagram for identifying samples containing sperm cells, and for isolating and analyzing captured sperm cells, according to some embodiments.

FIGS. 2A-B show schematic illustrations of complementary metal-oxide semiconductor (CMOS)-integrated (FIG. 2A) and smartphone-integrated (FIG. 2B) microfluidic systems for shadow imaging, capturing, and analyzing sperm cells, according to some embodiments.

FIG. 3A shows the monitoring, via shadow imaging, of sperm cells captured and isolated in a microfluidic platform, according to some embodiments.

FIG. 3B shows example microscope images of various types of sperm cells, and identification thereof, according to some embodiments.

FIGS. 4A-B show an example microfluidic device with microchannels for selective sperm capture, isolation, detection and quantification, according to some embodiments.

FIG. 5 shows an example detailed view of the capture of sperm cells utilizing sialyl-Lewis^{x} sequence (SLeX) oligosaccharide, according to some embodiments.

FIG. 6A shows a schematic illustration of capture of sperm cells in the microchannels of a microfluidic device in the presence of a blocking agent, according to some embodiments.

FIGS. 6B-C illustrate the effect of a blocking agent and SLEX concentration in the sperm capture efficiency of a microfluidic device, according to some embodiments.

FIG. 7 shows an aspect of surface chemistry for capture of sperm cells utilizing sialyl-Lewis^{x} sequence (SLeX) oligosaccharide, according to some embodiments.

FIGS. 8A-B show example results of differential extraction of sperm and/or epithelial cells, according to some embodiments.

FIGS. 9A-D show an example differential extraction process of aged sperm cells to isolate various types of sperm cells from epithelial cells, according to some embodiments.

### DETAILED DESCRIPTION OF SOME OF THE EMBODIMENTS

Embodiments to a sperm cell capture system and methodology (together or separately referred to as “platform(s)” or “system(s)”) for direct and intact sperm cell detection and/or isolation using the inner surface(s) microfluidic channels are disclosed herein. In some embodiments, the platforms provide expedited testing for forensic as well as hospital and primary care settings. Moreover, in some embodiments, label-free, bio-detection functionality such as electrical, mechanical and optical mechanisms (including photonic and plasmonic) can be used for the monitoring, detection, capture, isolation, and/or quantification of sperm cells from bodily and clinically relevant fluids. In some embodiments, conjugated magnetic beads may be used (in addition to or in place of the surfaces of microfluidic channels) for capturing sperm cells. In some embodiments, detection for multiple morphologies of sperm cells ranging from forensic applications to laboratory research, medical diagnostics and drug development/treatment are provided.

In some embodiments, a detection method is provided and includes flowing a biological sample within one or more microfluidic channels so as to capture sperm cells and perform shadow imaging using, for example, holographic algorithms (also including other static and dynamic imaging algorithms) with the microfluidic platform to obtain one or more images of bound sperm cells (for example). Some embodiments of holographic imaging are discussed in the publication by Sobieranski et al., Light: Science & Applications 4, e346; doi:10.1038/lsa.2015.119 (2015), entitled “Portable Lensless Wide-field Microscopy Imaging for Health-Care Applications using Digital In-line Holography and Multi-Frame Pixel Super-Resolution,” the content of which is incorporated herein by reference in its entirety.

In some embodiments, a plurality of capture reagents may be used to isolate target particles (e.g., cells, molecules) and/or target analytes from forensic samples containing such target particles. In particular, and for example, embodiments of microchips (e.g., forensic microchips) described herein may be used to capture target cells/analytes with high efficiency and specificity. Surfaces having the captured cells/analytes can then be monitored under, for example, an optical shadow imaging means which may be used with one or more algorithms such as holographic algorithms as well as other static and dynamic imaging algorithms for label-free detection and quantification. In some embodiments, the combination of such microchip embodiments with a lens-less imaging system (i.e., shadow imaging) provides for a portable and optionally battery-powered capture and detection system which can be used I the field (for example). Such embodiments are configured to overcome many of the deficiencies with existing technologies, which are limited by required equipment, time, cost, and other processing factors. In come embodiments, a shadow imaging platform integrated with a microfluidic device is provided, which includes an inlet for reception of a biological sample. The inlet is in fluid communication with one or more microfluidic channels, each having at least one surface configured for capture detection with one or more capture reagents.

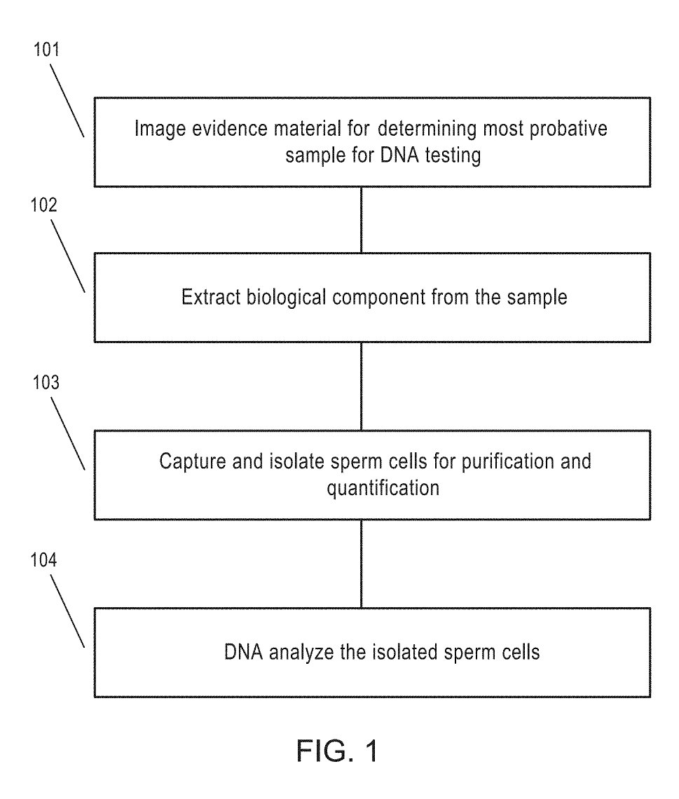

FIG. 1 is an example flow diagram for at least one of identifying samples containing sperm cells, and isolating and/or analyzing the captured sperm cells. Initially, samples which are hoped to contain biological materials (which may be referred to as a biological sample according to embodiments of the disclosure) from evidence material may be extracted using several methods. For example, pieces of evidence material samples (e.g., cotton swap or gauze pieces) may be eluted in phosphate buffered saline (PBS) (e.g., 500 μL of 1×PBS) and placed in a low temperature mixer (e.g., 4° C. thermomixer) set at a high rpm (e.g., about 1000 rpm) for a set amount of time (e.g., about 1 hour). The pieces may then be removed and placed in spin baskets that are subsequently centrifuged for short period of time (e.g., about 5 minutes) to pellet the solids in the solution. Some of the 1×PBS (about 300 μL) may then be removed without disturbing the pellet, which may be suspended by pulse vortexing.

After obtaining one or more biological samples, at step 101, such samples suspected of containing target cells (e.g., sperm cells) may be screened to identify probative samples that are candidates for further analysis (for isolating and study sperm cells contained within the samples). Such embodiments are highly advantageous compared to prior devices/methodologies where only few of collected samples are screened due to the long period of time to perform DNA testing. Prior devices/methodologies are often a waste of time and resources as most samples do not contain viable sperm cells for analysis.

Thus, in sharp contrast to the prior art, some of the embodiments of the disclosure allow for preliminary testing of samples (and in some embodiments, at the crime scene or hospital) for the presence of sperm. Such embodiments, include, for example, a rapid imaging via a microfluidic chip/cartridge that initially detects the presence of sperm to identify the probative samples for further analysis (e.g., DNA analysis). An exemplary method embodiment of such detection includes inputting the sample (or a portion thereof) suspected of containing sperm cells into a microfluidic platform. After the sample/portion thereof is positioned (e.g., flowed) within the one or more channels, imaging may be performed (i.e., shadow imaging) on the sample/portion to ascertain whether the sample contains sperm cells. Other methods of detecting presence of sperm cells include direct microscope analysis, use of chemical stains, and/or the like. Details on at least the use of staining methods are discussed in Allery et al., J. Forensic Science 46(2): 349-351 (2001), entitled “Cytological Detection of Spermatozoa: Comparison of three staining methods,” the content of which is incorporated herein by reference in its entirety.

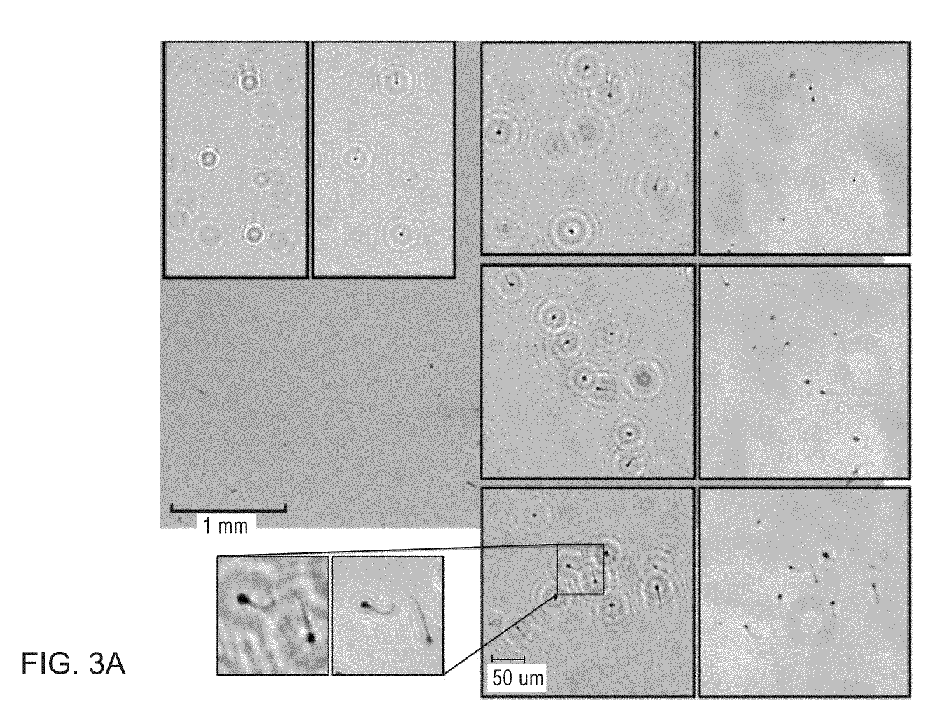

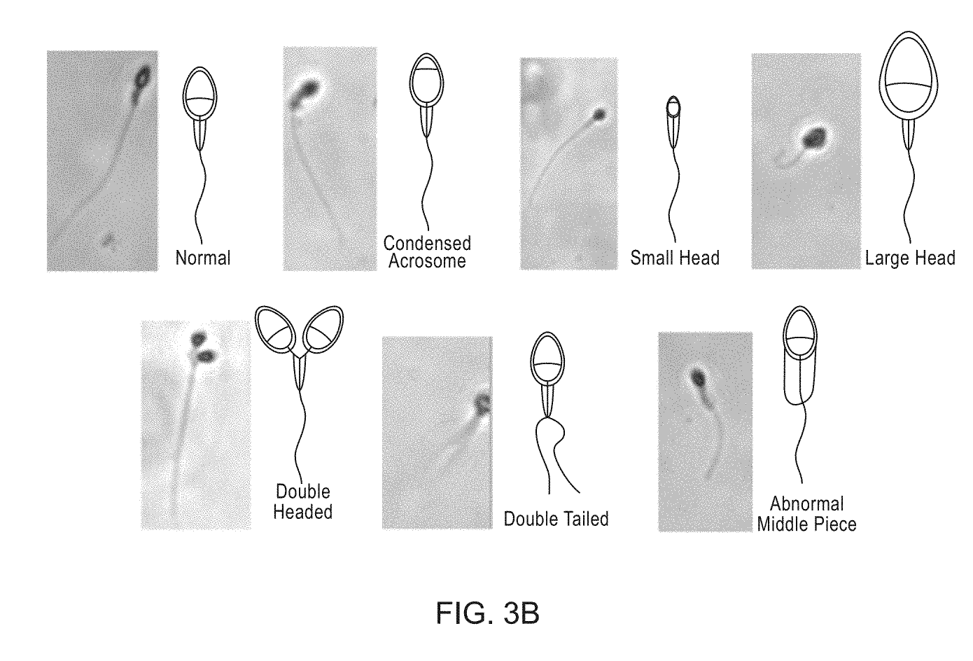

In some embodiments, for example, the imaging comprises shadow imaging. Such shadow imaging can utilize holographic, static and/or dynamic algorithms so as to help obtain images of sperm cells in the sample/portion. In some cases, such imaging can not only identify sperm cells, but also provide further details on captured sperm cells, such as but not limited to, the quantity of the sperm cells, which may allow for the determination of the more probative samples that can be used for additional focused DNA testing. For example, the imaging may provide a broad range of different morphologies of sperm cells. In some embodiments, other imaging techniques may also be used. For example, microscope imaging may be used to obtain a broad range of different morphologies of sperm cells as presented in FIG. 3B. Analysis of these images may allow a laboratory technician to identify the above noted probative samples.

Upon the selection of the probative samples for DNA analysis, in some embodiments, the same or new (and/or different) microfluidic platform may be utilized to extract biological components (step 102) from the samples, thereafter, sperm cells are captured (step 103). Such captured sperm cells can then be further analyzed (step 104) for DNA analysis (for example).

In some embodiments, the extracted biological components may now contain epithelial as well as sperm cells, and one may desire to isolate the sperm cells for analysis, e.g., step 103. For example, for samples derived from crime scenes such as sexual assaults, sperm cells from a perpetrator, epithelial cells primarily from the victim but also some from the perpetrator, and perhaps some DNA resulting from lysed cells may occur in the biological components. Depending on the specifics of the case, there may be multiple contributors of the epithelial and sperm cells. In some embodiments, the epithelial cells may be lysed, and the resulting mixture may flow through the device, which may result in the collection of sperm cells present and accounting of the sperm cells by a holographic imaging system.

In some embodiments, the lysis of the epithelial cells may occur after the capture of the sperm cells on the microfluidic surface or any solid surface to which the capture moiety has been linked. The lysed or unlysed epithelial cells along with free DNA and other components of the biological sample may then be collected and could be retained if there is a desire to analyze the DNA of the material based on the specifics of the forensic case.

At step 104, in some embodiments, the sperm cells are now separated and purified from the other components in the biological components, and the sperm cells may be eluted from the microfluidic platform (or bead or micro titre plate or any insoluble substrate) if there is a reversible linker present in the sperm attachment moiety or the sperm may be lysed on the substrate to release the DNA which can then be isolated for subsequent analysis.

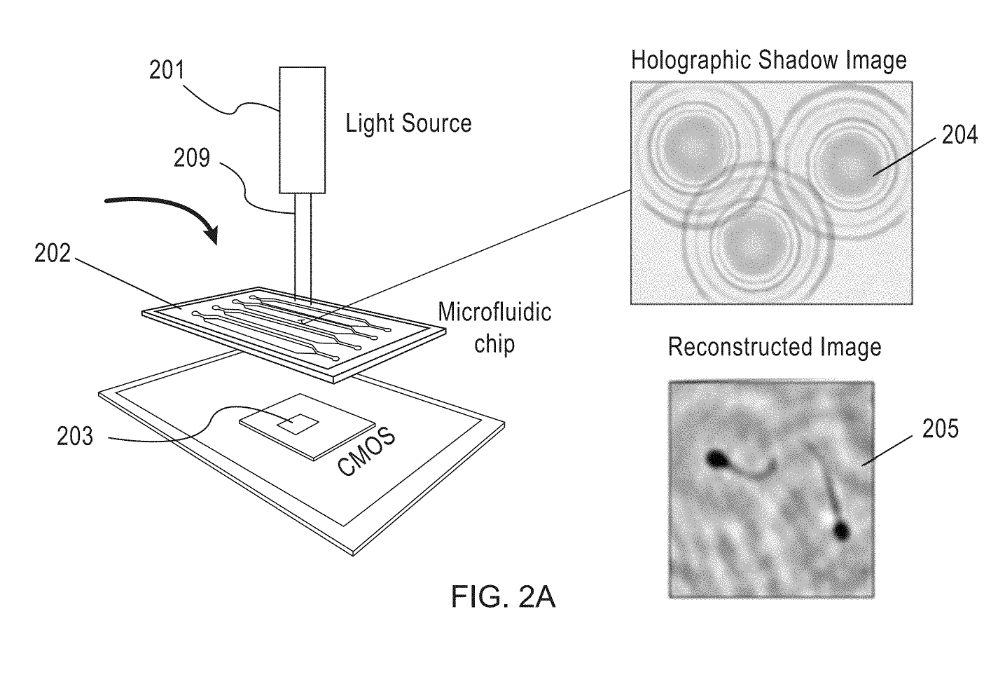

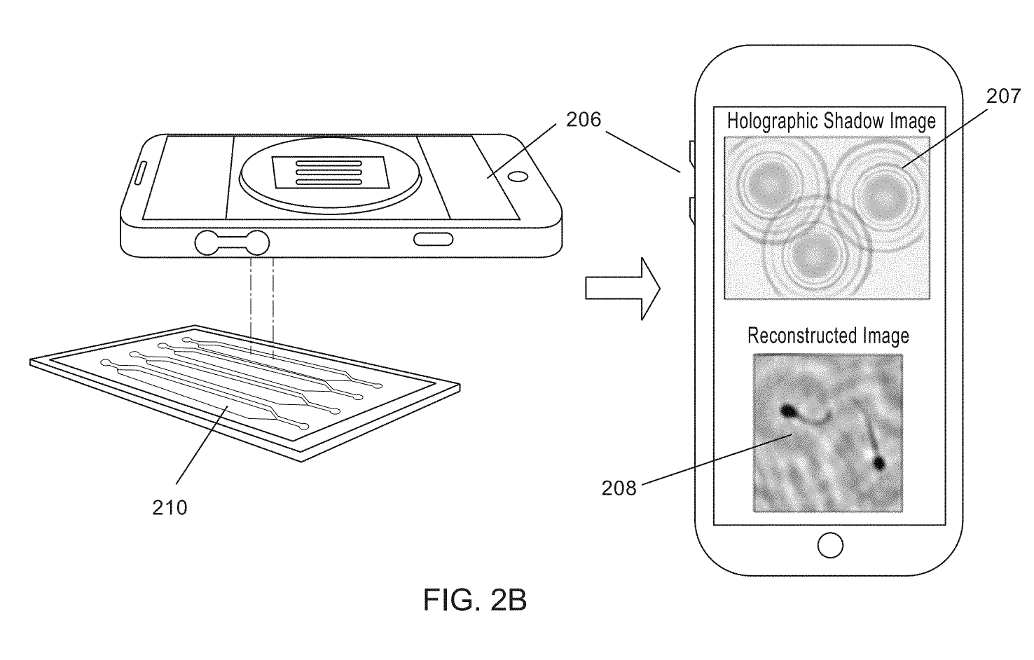

With reference to FIG. 2, in some embodiments, shadow imaging means (configured such that it does not require pre-labeling of a sample) can be utilized for sperm cell imaging/visualization. In some embodiments, a microfluidic chip is provided which includes a sperm capturing reagent such as, but not limited to, sialyl-Lewis^{x} sequence (SLeX), which may be employed along. The imaging means (e.g., shadow imaging detector) can be integrated with different algorithms such as holographic as well as other static and dynamic imaging algorithms. Moreover, in such embodiments, the imaging means may also include LED illumination and a CMOS image sensor. The reagent is configured to capture (and thus, separate) sperm cells from epithelial cells (e.g., in sexual assault evidence). Such a microfluidic process can also allow for quantification of sperm cells bound to a channel(s) in the chip, and thus, can be used to identify the probative samples themselves (and effective analytical methods for forensic analysis thereafter).

FIGS. 2A-B provide schematic illustrations of complementary metal-oxide semiconductor (CMOS)-integrated (FIG. 2A) and smartphone-integrated, for example (FIG. 2B), microfluidic systems that can be used to at least one of initially identify the probative samples, and/or to shadow image, capture, and/or analyze sperm cells contained in the samples. In FIG. 2A, light 209 from a light source 201 may be shone onto a microfluidic chip/cartridge 202 with CMOS image detector. The cartridge/chip comprises microfluidic channels upon which probative samples are provided therein (e.g., via flow) that include sperm cells. In some embodiments, a shadow image 204 (e.g., holographic) of the sperm cells contained in the probative samples may be obtained by via the CMOS image sensor (or other sensor; e.g., CCD sensor). The holographic shadow image 204 may further be processed (e.g., via holographic, static, dynamic, and/or the like imaging algorithms) to produce a reconstructed image 205 of the sperm cells in the samples. Features of this shadow imaging means (which are generally lens-less) have been discussed in the article by Zhang et al., entitled “Lensless imaging for simultaneous microfluidic sperm monitoring and sorting,” in the publication Lab on a Chip, issue 15, vol. 11, pp. 2535-2540 (2011), and in PCT Publication No. WO/2014/047608, entitled “Portal and Method for Management of Dialysis Therapy,” the entire contents of both of which are expressly incorporated by reference herein.

In some embodiments, in place of or in addition to a microfluidic cartridge/chip-based shadow imaging system, a smartphone-integrated microfluidic system may be used for studying the probative samples. FIG. 2B shows a smartphone 206 capturing data (e.g., image) from a microfluidic cartridge/chip 210 comprising a sample that contains sperm cells. For example, if the smartphone contains a CMOS chip, then the smartphone may be attached to the shadow imaging device to record the image of sperm cells. In some embodiments, the image data may include information that allows an application operating on the smartphone to identify the sperm cells and some or all of the associated properties thereof. For example, the data may include contrast, color, sharpness, hue, shadow etc., information that allows the application to determine the type, size, etc., of the sperm cells being studied (as well as the number of sperm cells). Accordingly, from such data, in some embodiments, the application can produce a shadow image 207 (e.g., holographic) from which a reconstructed image 208 of the sperm cells can be created. For example, images obtained by the smartphone may be analyzed using holographic algorithms (e.g., a holographic software) to determine the presence or absence and in some cases quantity of sperm cells in the sample. In some embodiments, the images may not be collected by the smartphone, but they may be received by an external server that, by using the holographic algorithms, processes the images so as to determine the presence/absence and additionally quantity of the sperm cells in the samples. Further, the results may be received by the smartphone (e.g., via wired or wireless (e.g., wifi, Bluetooth, etc.) connections) from the server. The use of a smartphone is particularly beneficial in that the initial screening of evidence materials to determine a probative sample for further analysis and DNA testing (or inclusion into a rape kit, depending on the circumstance) can be performed on location (e.g., at a crime scene, hospital, etc.) relatively rapidly and conveniently given the portability of smart phones (e.g., compact, mobile computing/imaging devices).

FIG. 3A shows shadow images of sperm cells captured and isolated in a microfluidic platform, according to some embodiments. These images allow for the monitoring of the captured sperm cells, allowing one to identify a broad morphology of sperm cells with applications not only in DNA forensics but also in laboratory research, medical diagnostics, drug development/treatment, and/or the like. For example, from the study of shadow images such as the ones depicted in FIG. 3A, in some embodiments, one may identify several forms of sperm cells, including normal sperm cells and sperm cells with condensed acrosome, small heads, large heads, double heads, doubled tails and an abnormal middle-piece, e.g., FIG. 3B.

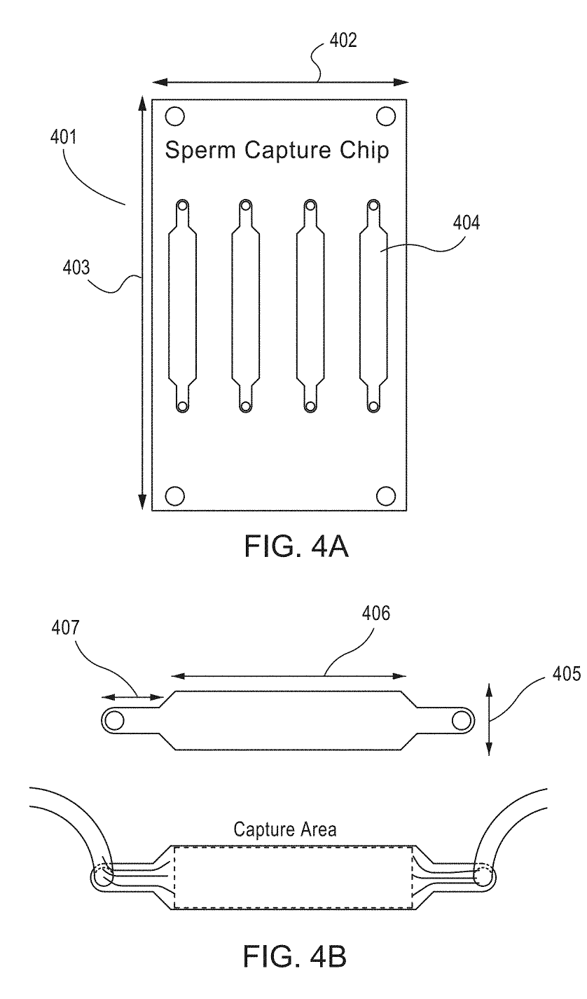

With reference to FIG. 4A, in some embodiments, a microfluidic device 401 for selective sperm cell capture, isolation, detection and quantification is shown (at least one thereof). In some embodiments, the device 401 may be fabricated without utilizing photolithographic methods or a clean room. The device 401 may be constructed so as to have a plurality of microfluidic channels 404 (one or more). For example, as shown in the embodiment of FIG. 4A, the microfluidic device 401 may include four parallel microfluidic channels within an area measuring about 40 mm in length 403 and about 24 mm in width 402.

FIG. 4B provides a schematic diagram of a microfluidic channel 404 comprising three regions, an inlet and an outlet, e.g., 407, and a capture area 406 where the capture and isolation of the sperm cells take place. In some embodiments, the capturing of sperm cells in the capture area 406 is facilitated by the differences in the dimensions of the lateral diameter 405 of the microchannel 404 and that of the inlet and/or the outlet 407. For example, the lateral diameter 405 may measure about 2.5 mm while that of the openings may measure about 1.53 mm. Further, the much larger length of the capture area (e.g., about 13.5 mm) also facilitates the capture and isolation of the sperm cells.

To construct the device, in some embodiments, poly(methyl methacrylate) (PMMA) (1.5 mm thick, McMaster Carr, Atlanta, Ga.) and double-sided adhesive (DSA) film (80 μm thick, iTapestore, Scotch Plains, N.J.) are fabricated using a laser cutter (Versa Laser™, Scottsdale, Ariz.). The inlets and outlets 407 at each end of the channels 404 are configured on the PMMA layer, and glass cover slips can then assembled using the DSA. To clean the chip base, the glass cover slip can be sonicated for about 15 min in ethanol. Following the cleaning step, the cover slip is then washed with distilled water and dried under nitrogen gas. To modify the surface, both sides of the glass cover slip can be plasma-treated for about 90 seconds. Then, PMMA, DSA, and glass cover slip can be assembled to produce the microfluidic device.

In some embodiments, the substrate of the sperm capture area/region can be optically transparent to facilitate shadow imaging and optical measurement. Thus, polystyrene, glass parylene, quartz crystal, graphene and mica layers, and poly(methyl methacrylate) can be used for the substrate. These materials are optically transparent and are capable of supporting the functionalization of the surfaces of the capture area 406, which selectively bind to sperm cells via surface recognition elements linked thereto (e.g. reagent) such as specific saccharides units and antibodies and which possess the optical properties for the monitoring of the binding and capture events.

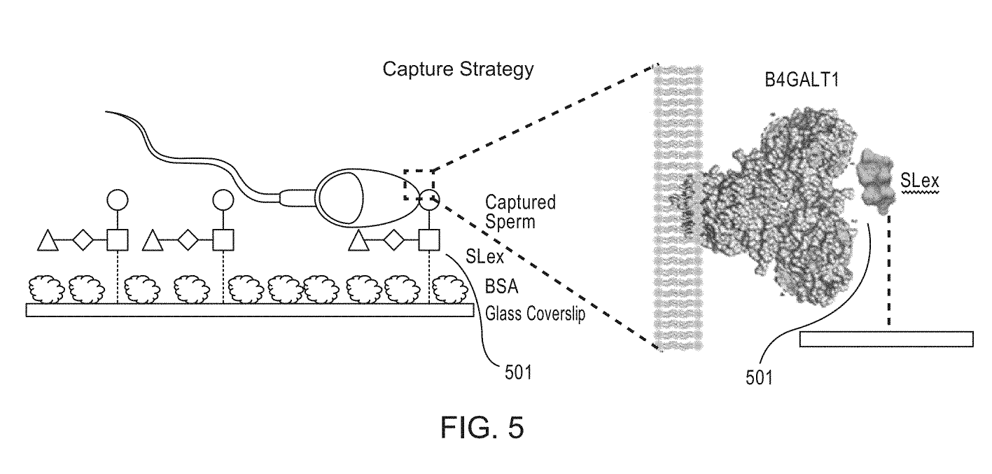

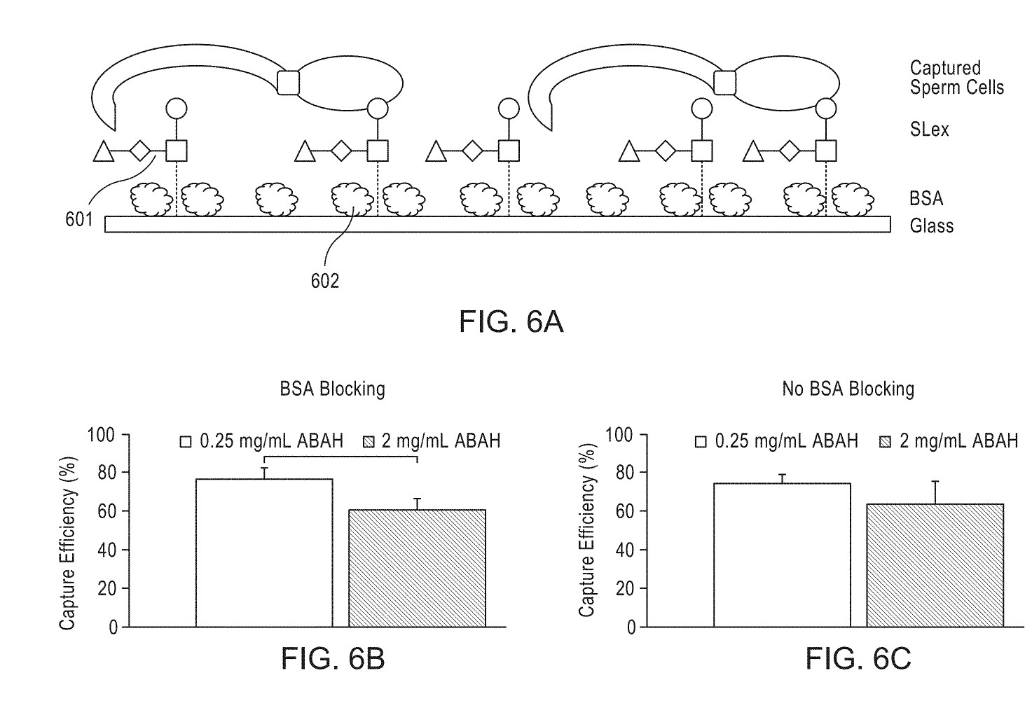

In some embodiments, with reference to FIG. 5, the capturing of sperm cells in the capture area 406 of the microchannels 404 may be accomplished via capturing reagents such as, but not limited to, oligosaccharides that may be utilized for the processing of forensic biological samples. An example of such capture reagents is a unique oligosaccharide (i.e., SLeX sequence [NeuAca2-3Galβ1-4(Fucα1-3)GlcNAc]) 501 located on the extracellular matrix (i.e., zona pellucida (ZP)) of the oocyte. This oligosaccharide sequence is an abundant terminal sequence on human ZP that represents a ligand for human sperm-egg binding. In some embodiments, the oligosaccharide SLeX agent captures sperm cells by binding to the B4GALT1 (beta1-4galactosyltransferace 1) gene on sperm cells. Discussion of SLeX and its role in sperm-egg binding have been presented in the article by Pang et al., entitled “Human Sperm Binding Is Mediated by the Sialyl-Lewis^{x} Oligosaccharide on the Zona Pellucida,” in the publication Science, 333, 1761 (2011), the entire content of which is expressly incorporated by reference herein. In some embodiments, utilizing such capture reagents, a disposable microfluidic chip that can detect and capture sperm cells from unprocessed bodily fluids can be developed. For example, microfluidic channels that are functionalized using salinization-based surface chemistry may contain immobilized SLeX oligosaccharide 501 that can be used to selectively capture sperm cells. SLeX oligosaccharide 501 has a great advantage over antibody-based methods in that it possesses long shelf life and storage capability. Thus, the disclosed separation and detection platform can allow efficient separation of sperm cells from epithelial cells in sexual assault evidence materials, reducing analysis time and accelerating the forensic process. Specific sperm capture can also be achieved through the use of antibodies, as discussed below with reference to FIG. 6, for example.

In some embodiments, other mechanisms that facilitate the formation of sperm-egg fusion may also be used to capture sperm cells. For example, equatorial segment protein 1 (ESP1), a testis specific protein, has been shown to be highly conserved and to play a key structural role during the fusion of a sperm cell with an egg. As such, any molecule on the oocyte that binds to ESP1 can be used as a capture agent in a similar manner as SLeX oligosaccharide. Some discussion of ESP1 and its role in sperm-egg binding have been presented in the article by Suryavathi et al., entitled “Dynamic Changes in Equatorial Segment Protein 1 (SPESP1) Glycosylation During Mouse Spermiogenesis,” in the publication Biology of Reproduction, vol. 92, no. 5, 129 (2015), the entire content of which is expressly incorporated by reference herein.

In some embodiments, with reference to FIG. 6A, the immobilization of the SLeX oligosaccharide 601 and/or antibodies in the microfluidic channels so as to facilitate the capture of sperm cells may be enhanced by a modified support surface. For example, to link the one or more surface recognition elements such as specific saccharide units, antibodies, etc., into the microchannels, a modified support surface may be formed by a 3-mercaptopropyl-trimethoxysilane (3-MPS) to form thiol groups, reacting N-(gammamaleimidobutyryloxy) succinimide ester (GMBS) with the succinimdie groups to form an amine reactive intermediate, and stabilizing the amine reactive intermediate by 4-Aminobenzoic acid hydrazide (ABAH) to form the modified support surface. For example, a glass slide can be modified with oxygen plasma (100 mW, 1% oxygen) for about 90 seconds in a PX-250 chamber, followed by a silanization step using about 200 mM of 3-mercaptopropyl-trimethoxysilane (3-MPS) dissolved in ethanol. After the silanization step, the glass slide can be assembled with a PMMA-DSA construct to form a microfluidic channel. Further, N-(gammamaleimidobutyryloxy) succinimide ester (GMBS) can be used as an amine reactive intermediate, and after GMBS incubation, the surfaces can be stabilized using an 4-Aminobenzoic hydrazide (ABAH) to create binding groups for SLeX. In some embodiments, to minimize the unspecific binding of cells, a blocking agent 602 may also be employed into the microchannels. The modified support surface may be linked to a SLeX material 601 for the capture of sperm.

For example, with reference to FIGS. 6B and 6C, in some embodiments, the results of experiments conducted where a blocking agent (e.g., Bovine serum albumin (BSA), about 1%) was applied into the microchannels to minimize unspecific binding of cells, and incubated for 30 minutes at 4° C. are shown. Before sampling sperm cells, the channels were washed out with PBS a few times again (e.g., about three times). After surface chemistry steps, sperm samples (˜1,000-5,000 cells/mL) were applied into the channels, and incubated for about 30 minutes at room temperature, followed by microscopy imaging to quantify the sperm cell number in the microchannels. Further, the microchannels were washed with PBS using a syringe pump at about 5 μL/min for about 20 min, followed by microscopy imaging to evaluate the capture efficiency. In the experiments, two different SLeX concentrations and the effect of BSA blocking were evaluated. As a result, it was observed that 0.25 mg/mL of SLeX concentration provided statistically more sperm cell capture in microchannels (n=3-4, p<0.05), e.g., FIG. 6B. The BSA blocking step did not significantly change the capture efficiency of sperm cells when different concentrations were used, e.g., FIG. 6C. In some embodiments, a SLeX modified microfluidic chip may have approximately 77% capture efficiency for human sperm cells by coupling of 4-Aminobenzoic acid hydrazide (ABAH) with this specific carbohydrate unit.

For antibody-based capture events, NeutrAvidin protein, Protein A/G or Protein G may be used to immobilize specific antibodies. Additionally, other or additional antibodies may be present based on the sperm types to be detected. It is contemplated that multiple sperm types may be detected on a single platform. In some embodiments, the antibody may be a polyclonal or monoclonal antibody. Additionally, in some forms, the modified support surface is linked to at least one of a protein A, a protein G, a protein A/G, a Streptavidin protein, and a NeutrAvidin protein which is used to form chemical bonds and as well as physical adsorption to immobilize recognition elements such as the antibody on the modified support surface.

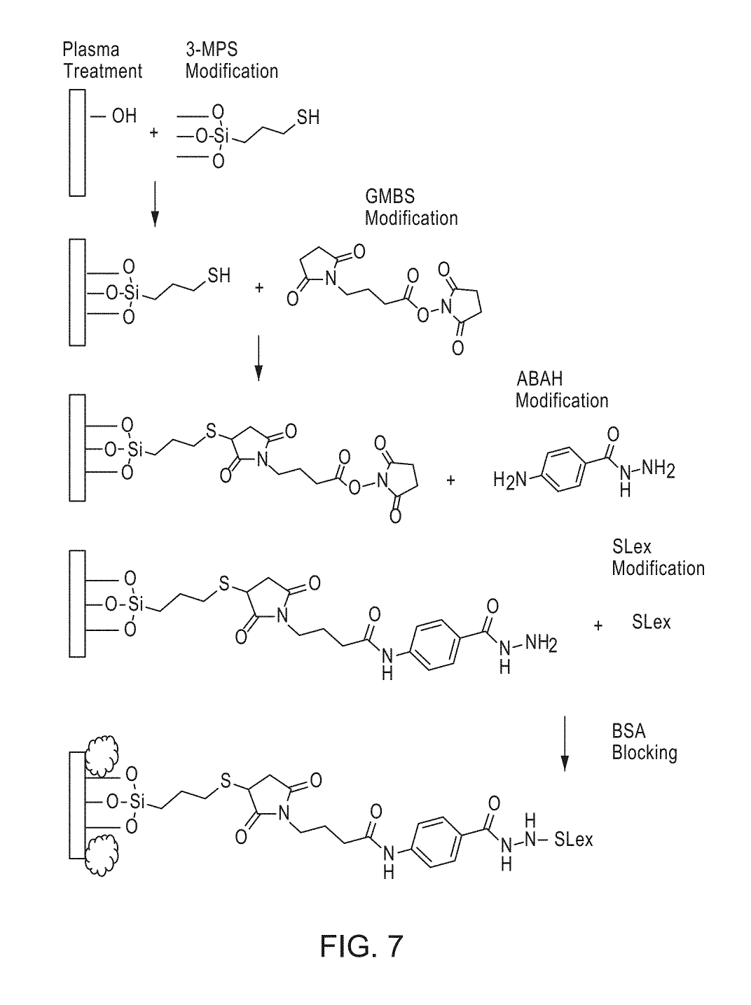

With reference to FIG. 7, in some embodiments, an example process of modifying the surface of one or more channels in the microfluidic cartridge/chip (e.g., with one or more chemicals. and SLeX molecules) to capture sperm cells is shown as an example. Accordingly, after plasma treatment and chip construction, 3-mercaptopropyl-trimethoxysilane (3-MPS) (e.g., about 200 mm in ethanol, about 100 μL) was passed through the channels and incubated for a period of time (e.g., in some embodiments, about 30 minutes) at room temperature. N-(gammamaleimidobutyryloxy) succinimide ester (GMBS) (e.g., about 4% in ethanol, about 30 μL) was then incubated in microchannels for about 45 minutes at room temperature. Hence, GMBS generated succinimide groups to bind amine-functionalized groups, ABAH molecule in this case. Between the chemical modification steps, the channels were washed out with ethanol and PBS (about 100 μL) to remove the excess of untreated reagents. To immobilize the sperm recognition element, two different concentrations of ABAH molecule (about 0.25 mg/mL and about 2.5 mg/mL) were evaluated by incubating for an hour at room temperature. The microchannels were then washed three times with PBS (about 100 μL), followed by an incubation of SLeX solution (about 100 μg/mL in PBS) overnight at 4° C. Then, the microchannels were washed with PBS three times again.

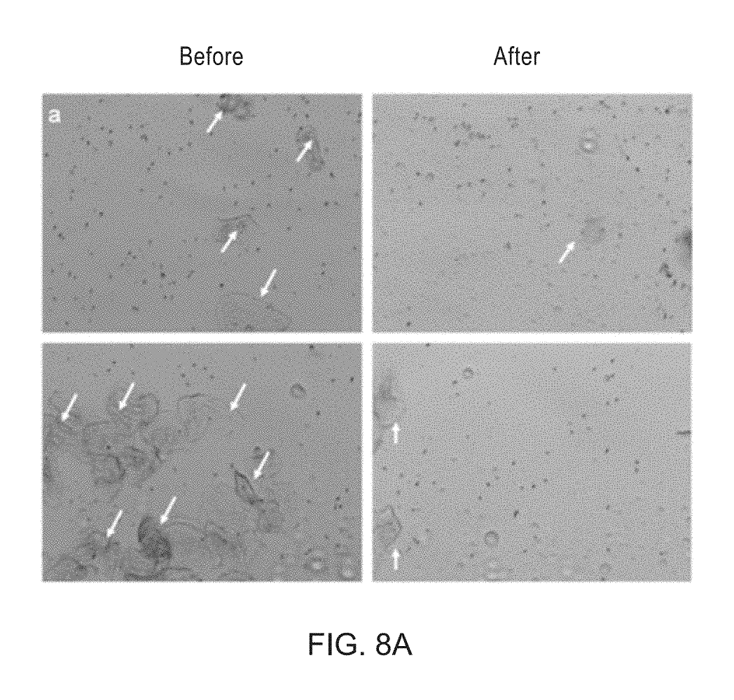

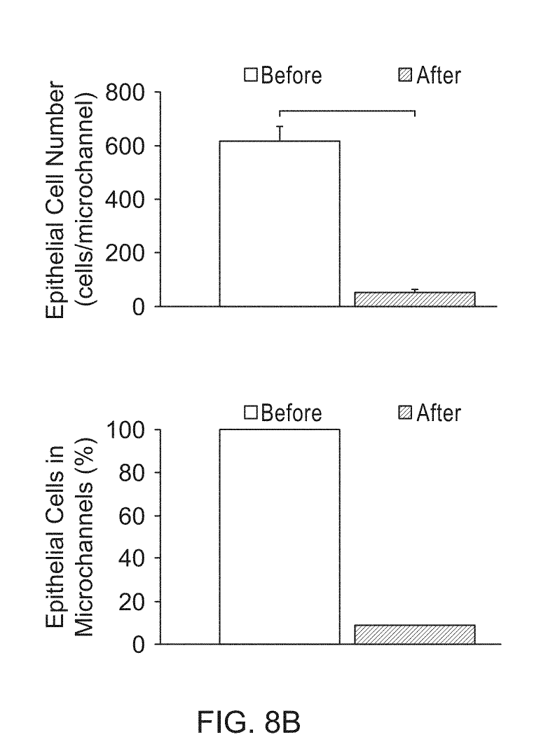

In some embodiments, with reference to FIG. 8, if desired, the sample may be eluted from the capture agents immobilized on the chip to recover the forensic evidence, or in the case of sperm cells, the cells may be lysed (for example, with enzymes and reducing reagents) and DNA recovered for further analysis such as downstream genomic analyses. Other biological entities such as epithelial cells will flow through the chip, and they can be recovered for further processing. Differential extraction processes may be used to allow for the extraction of DNA material from the sperm cells, and if needed from the epithelial cells, with little or no mixing between the DNAs from the different types of cells (i.e., with little or no mixing between the sperm cell DNA and the epithelial cell DNA). See, e.g., K. M. Horsemen, et al., “Separation of Sperm and Epithelial Cells in a Microfabricated Device: Potential Application to Forensic Analysis of Sexual Assault Evidence,” Anal. Chem. 2005, 77, pp. 742-749. For example, the lysis of sperm cells as described above may break down the membranes of the sperm cells, allowing for the extraction of DNA within. For example, chemicals such as dithiothreitol (DTT) may be used to disrupt the sulfur bonds in the coating of sperm cells, facilitating the extraction of the DNA. In some embodiments, standard DNA extraction techniques such as phenol/chloroform extraction and the like may then be utilized.

The shadow imaging platform may provide valuable information to the forensic analyst by quantifying sperm cells from the forensic sample, e.g., FIG. 8B. For example, samples containing 30-40 or more cells (approximately 90-120 pg DNA) may be targeted for standard short tandem repeat (STR) process with commercial kits while samples with less than 20-30 cells may be directed for less informative Y-STR analysis. Also, by comparing the sperm counts from multiple samples, the technology may be used to help direct the analyst to the most probative samples for investigation.

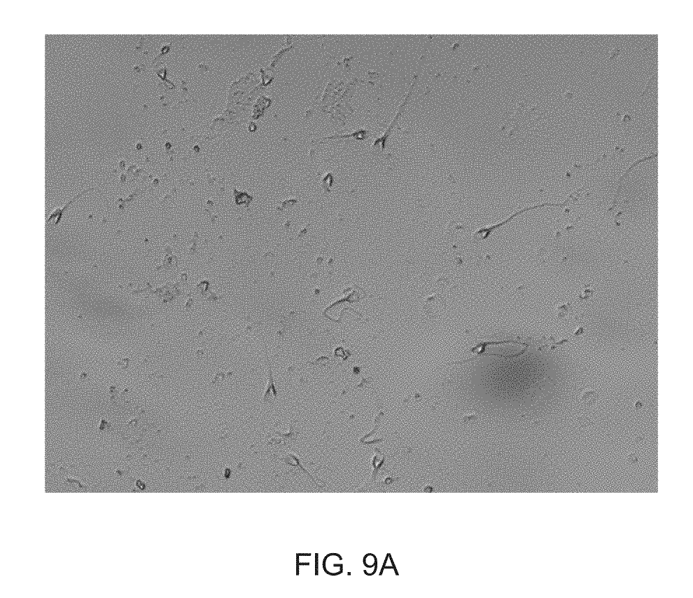

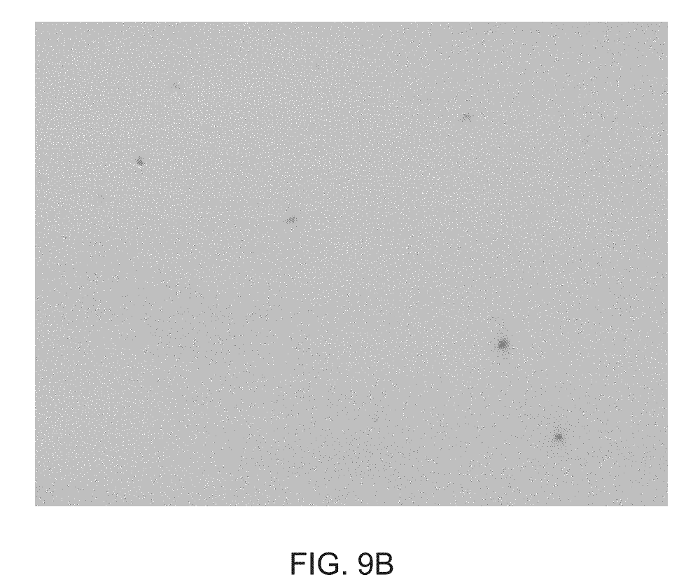

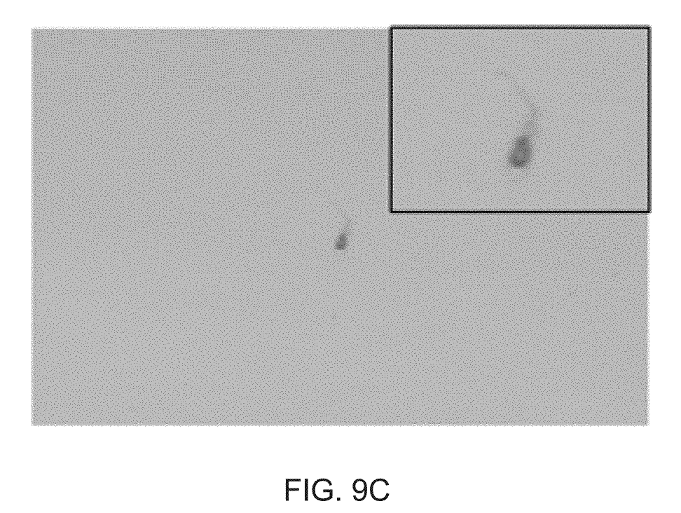

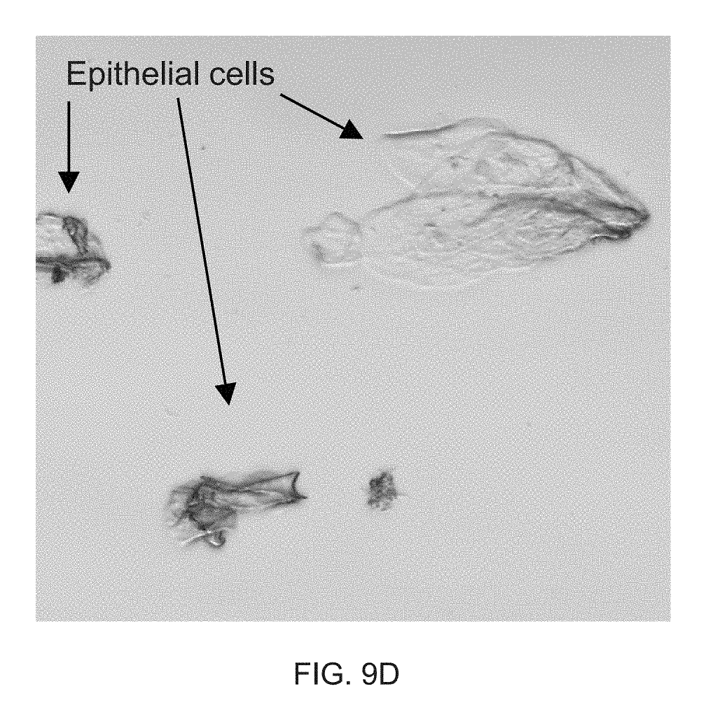

In some embodiments, FIG. 9 shows an example differential extraction process of aged sperm cells from epithelial cells as a demonstration of the capabilities of the platforms and systems disclosed herein. One notes that when aged sperm cells (FIG. 9A) are extracted from epithelial cells (FIG. 9D), most of them have some deformities such as missing tails (FIG. 9B) while a few of them retain their tails (FIG. 9C). Table 1 here shows the results of a differential extraction of aged forensic samples done using the apparatus, systems and methods of the present disclosure. The table shows that using the microfluidic platform of the present disclosure, in some embodiments, a high sperm capture efficiency may be obtained for a variety of samples. In some cases, the impurity level, i.e., the presence of epithelial cells, may be kept to a low level. Sperm capture efficiency may be defined as the ratio of the number of sperm remaining after washing of the sample (i.e., the captured sperm) compared to the number before the capture. In some instances, the impurity level can be measured by the ratio of the number of epithelial cells remaining after washing compared to the initial number of sperm cells, i.e., the number of pre-wash sperm cells. In the specific embodiment of Table 1, the capture efficiency for the samples described therein ranges from about 70% to about 93% while the impurity level ranges from about 6% to about 16%.

TABLE 1

| | | Isolation- | # of | # of Retained | Capture | |
|---|---|---|---|---|---|---|
| | Sample | Collection | Captured | Epithelial | Efficiency of | |
| Sample | Explanation | Material | Sperm | Cells | Sperm (%) | Impurity (%) |
| A | Post coital vaginal | ~⅓ of a | 685 ± 101 | 41 ± 11 | 92.4% | 6.1% |
| | swab with semen | cotton swab | | | | |
| B | Unknown Sample | Cotton gauze | 363 ± 136 | 55 ± 36 | 82% | 16.1% |
| C | Buccal cells mixed | N/A | 275 ± 52 | 58 ± 15 | 82% | 19.8% |
| | with semen | | | | | |
| E | Mixed semen on | Cotton swab | 661 ± 315 | 31 ± 19 | 86.3% | 6.7% |
| | cotton swab | | | | | |
| F | Mixed semen on | Cotton gauze | 289 ± 19 | 48 ± 33 | 70.1% | 16.1% |
| | cotton gauze | | | | | |

Thus, the microfluidics and shadow imaging (for example) means embodiments disclosed herein integrate multiple steps on a single device (e.g., compact, mobile device), improve the scaling capacity, which enables minimal reagent consumption, reducing the need for skilled analysts, etc. Such microfluidic-based embodiments incorporate flow and detection capabilities including optical, electrical and/or mechanical tools for the capture and sort of various type of cells and pathogens (e.g., sperm, white blood cells, bacteria, yeasts, fungi, microbes, and viruses) may be applied to problems of forensic investigation, among other applications.

Statistical Analysis. To evaluate ABAH concentrations, embodiments of the disclosure can employ one-way analysis of variance (ANOVA) with Tukey's posthoc test followed with Bonferroni's Multiple Comparison Test for equal variances for multiple comparisons with a statistical significance threshold set at 0.05 (p<0.05). Error bars in the plots represented standard error of the mean (SEM), and brackets demonstrated the statistical difference between the groups. GraphPad Prism (Version 5.04) was used in all statistical analyses.

Although the above discussion has been provided with respect to a microfluidic device, in some embodiments, all the features of the present disclosure, including the usage of the oligosaccharide SLeX sequence to capture and isolate sperm cells can be applied to non-microfluidic devices. For example, a well-type surface incorporating the oligosaccharide can be used for similar purposes of isolating and capturing biological materials such as sperm cells from biological samples. However, it is worth noting that the surface can be any geometry including a spherical surface (for example), and/or other 2D and 3D geometrical surfaces configured to capture a target (e.g., beads, magnetic beads).

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Some embodiments of the present disclosure may be distinguishable from one and/or another prior art reference by specifically eliminating one and/or another structure, functionality and/or step. In other words, claims to some embodiments of the inventive subject matter disclosed herein may include negative limitations so as to distinguish from the prior art.

When describing the sperm capture platform and binding of an antibody or other molecule thereto (in accordance with the various disclosed embodiments), terms such as linked, bound, connect, attach, interact, and so forth should be understood as referring to linkages that result in the joining of the elements being referred to, whether such joining is permanent or potentially reversible. These terms should not be read as requiring the formation of covalent bonds, although covalent-type bond might be formed.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for at least one of capturing and detecting target cells in a biological sample, comprising:
one or more microfluidic channels for receiving a biological sample;
a recognition reagent linked to a surface of the one or more channels, the reagent configured to capture one or more target cells contained in the biological sample by binding with the one or more target cells; and
monitoring means configured to at least one of monitor the surface and detect one or more captured target cells bound with the reagent linked to the surface.

2. The system of claim 1, wherein the monitoring means is additionally configured to at least one of receive and collect data on the captured target cells.

3. The system of claim 1, wherein the target cells in the biological sample are selected from the group consisting of: sperm cells, blood cells, bacteria, yeasts, fungi, and viruses.

4. The system of claim 1, wherein the target cells in the biological sample are sperm cells.

5. The system of claim 1, wherein the recognition reagent comprises an oligosaccharide sequence.

6. The system of claim 5, wherein the oligosaccharide sequence comprises a sialyl-Lewis' oligosaccharide sequence.

7. The system of claim 1, wherein the monitoring means comprises imaging means configured to acquire images of captured target cells.

8. The system of claim 7, wherein the image means comprises shadow imaging means.

9. The system of claim 1, wherein the monitoring means includes at least one of mechanical means, electrical means, optical means, photonic means, and plasmonic means.

10. The system of claim 1, wherein the monitoring means comprises a smartphone equipped with an application configured for receiving and/or collecting the data of at least one of the surface and any captured target cells.

11. The system of any of claims 1-10, wherein the monitoring means is additionally configured to include at least one of static, dynamic and holographic imaging algorithms.

12. The system of claim 1, wherein the microfluidic channels have dimensions ranging from about 25 micron to about 80 micron.

13. A method for at least one of capturing and detecting target cells in a biological sample, comprising:
providing a surface having linked thereto one or more oligosaccharide molecules, wherein the oligosaccharide molecules are configured to capture one or more target cells;
exposing the surface to a biological sample;
capturing one or more target cells contained in the sample, wherein a target cell is captured by binding with at least one of the oligosaccharide molecules; and
at least one of monitoring the surface and detecting the at least one captured target cell.

14. The method of claim 13, further comprising at least one of receiving and collecting data corresponding to at least one of the surface and captured target cells.

15. The method of claim 13, wherein the target cells are selected from the group consisting of: sperm cells, blood cells, bacteria, yeasts, fungi, and viruses.

16. The method of claim 13, wherein at least one of monitoring and detecting comprises receiving and/or collecting data associated with at least one of the surface and captured target cells bound thereon via the oligosaccharide.

17. The method claim 16, wherein the data is received and/or collected via a monitoring means.

18. The method of claim 13, wherein the oligosaccharide molecules comprise sialyl-Lewis' oligosaccharide molecules.

19. The method of claim 13, wherein exposing comprises flowing the biological sample over the surface.

20. The method of any of claim 14, wherein at least one of receiving and collecting data comprises imaging the surface and/or bound target cells.

21. The method of any of claims 13-20, wherein the surface comprises at least one of the inner surface of one or more microfluidic channels and the surface of one or more beads.

22. The method of any of claims 13-21, further comprising at least one of releasing, lysing, and processing the captured target cells.

23. The method of claim 20, wherein imaging comprises acquiring shadow images of bound target cells.

24. The method of claim 13, wherein prior to exposing the surface to the biological sample, the method comprises identifying the biological sample as having probative value.

25. The method of claim 20, wherein identifying the biological sample having probative value comprises identifying biological samples containing target cells with determined morphology.

26. The method of claim 13, wherein the target cells are sperm cells.

27. A method for at least one of capturing and detecting target cells in a plurality of biological samples, comprising:
identifying, via shadow imaging, probative samples for capturing and detecting target cells from the plurality of biological samples;
providing a surface having linked thereto one or more oligosaccharide molecules, wherein the oligosaccharide molecules are configured to capture one or more target cells;
exposing the surface to the probative samples for capturing and detecting target cells;
capturing one or more target cells contained in the probative sample, wherein a target cell is captured by binding with at least one of the oligosaccharide molecules; and
differentially extracting DNA of the one or more target cells contained in the probative sample.
